(19) United States
(12) Patent Application Publication
(10) Pub. No.: US 2021/0290711 A1
(43) Pub. Date: Sep. 23, 2021

(54) HEMPSEED OIL COMPOSITIONS

(71) Applicant: Melissa Johnson, Chesterfield, MO (US)

(72) Inventor: Melissa Johnson, Chesterfield, MO (US)

(21) Appl. No.: 17/059,906

(22) PCT Filed: May 31, 2019
(86) PCT No.: PCT/US2019/035078

§ 371 Date: Nov. 30, 2020

Related U.S. Application Data
(60) Provisional application No. 62/679,288, filed on Jun. 1, 2018.

(51) Int. Cl. A61K 36/185; A23D 9/00; A23D 9/04; A23L 33/00; A23L 33/115; A23K 20/158; A61P 29/00

(52) U.S. Cl. CPC A61K 36/185; A23D 9/00; A23D 9/04; A23V 2002/00; A23L 33/115; A23K 20/158; A61P 29/00; A23L 33/40

(57) ABSTRACT

The invention disclosed herein generally relates to compositions including hempseed products such as a hempseed powder or oil, modified hempseed, and/or hempseed product extract(s); and methods for making and using the same.

### FIELD OF THE INVENTION

The invention disclosed herein generally relates to hempseed oil compositions and methods for making and using the same.

### BACKGROUND OF THE INVENTION

Hempseed oil is obtained from the seeds of Cannabis sativa L. Unmodified hempseed oil has an ideal omega-6 to omega-3 ratio and has been used for various nutritional benefits.

### SUMMARY OF INVENTION

Embodiments of the invention relate to compositions including hempseed products such as a hempseed powder or oil, modified hempseed product (hempseed product with one or more of its natural components removed, added or both), and/or hempseed product extract(s); and methods for making and using the same.

### DETAILS OF INVENTION

### Compositions

Compositions including or made from hempseed product are provided. The composition can include about 10%, 20%, 30%, 40% 50%, 60%, 70%, 80%, 90%, 95%, 99% or up to nearly 100% hempseed product, modified hempseed powder or oil, or hempseed powder or oil extract(s). Modified hempseed product as disclosed herein is defined by hempseed powder or oil with some or all of one or more of a natural component removed, some or all of one or more of a natural component added, or some or all of one or more of a natural component removed and some or all of one or more of a natural component added. Hempseed product extracts as disclosed herein are defined by one or more component extracted from hempseed product.

The composition can include hempseed powder or oil that is modified by changing the amount of a natural component of hempseed powder or oil Modified hempseed powder or oil can have one or more one or more of its natural components removed, one or more of its natural components added, or at least one component added and at least another component removed. The one or more natural components can be selected from one or more of the following groups: lipids, proteins, carbohydrates, minerals, vitamins, cannabinoids, and other molecules. The percentage of the natural component that is removed can be about 10%, 20%, 30%, 40% 50%, 60%, 70%, 80%, 90%, 95%, 99% or up to nearly 100% of the amount that is naturally occurring. The percentage of the natural component that is added can be about 10%, 20%, 30%, 40% 50%, 60%, 70%, 80%, 90%, 100%, 150%, 200%, 300% or more of the amount that is naturally occurring. The modification to the hempseed powder or oil can contribute to at least one beneficial characteristic of the composition. The modification to the hempseed powder or oil can have at least one more beneficial characteristic compared to an unmodified hempseed powder or oil composition or a different hempseed powder or oil composition. The addition of the one or more natural components can act synergistically with the hempseed powder or oil.

The following Table includes the approximate amounts of natural components of unmodified hempseed. The amount of any of the following components can be modified. In some embodiments of the invention, the amount of one or more the following components can be modified. The amount of the one or more components can be removed by about 10%, 20%, 30%, 40% 50%, 60%, 70%, 80%, 90%, 95%, 99% or up to nearly 100% or added by about 10%, 20%, 30%, 40% 50%, 60%, 70%, 80%, 90%, 100%, 150%, 200%, 300% or more of the amount listed in the following Table.

TABLE 1
Components of Hemp seed

| | | 1 Value | Data | Std. | |
|---|---|---|---|---|---|
| Nutrient | Unit | per 100 g | points | Error | 3.0 tbsp = 30.0 g |
| Proximates | | | | | |
| Water | g | 4.96 | 17 | 0.17 | 1.49 |
| Energy | kcal | 553 | — | — | 166 |
| Energy | kJ | 2313 | — | — | 694 |
| Protein | g | 31.56 | 17 | 1.53 | 9.47 |
| Total lipid (fat) | g | 48.75 | 17 | 1.78 | 14.62 |
| Ash | g | 6.06 | 11 | 0.43 | 1.82 |
| Carbohydrate, | g | 8.67 | — | — | 2.6 |
| by difference | | | | | |
| Fiber, total | g | 4 | 2 | — | 1.2 |
| dietary | | | | | |
| Sugars, total | g | 1.5 | 2 | — | 0.45 |
| Sucrose | g | 0.85 | 1 | — | 0.26 |
| Glucose | g | 0.2 | 1 | — | 0.06 |
| (dextrose) | | | | | |
| Fructose | g | 0.31 | 1 | — | 0.09 |
| Lactose | g | 0.07 | 1 | — | 0.02 |
| Maltose | g | 0.07 | 1 | — | 0.02 |
| Minerals | | | | | |
| Calcium, Ca | mg | 70 | 2 | — | 21 |
| Iron, Fe | mg | 7.95 | 2 | — | 2.38 |
| Magnesium, Mg | mg | 700 | 1 | — | 210 |
| Phosphorus, P | mg | 1650 | 1 | — | 495 |
| Potassium, K | mg | 1200 | 1 | — | 360 |
| Sodium, Na | mg | 5 | 2 | — | 2 |
| Zinc, Zn | mg | 9.9 | 1 | — | 2.97 |
| Copper, Cu | mg | 1.6 | 1 | — | 0.48 |
| Manganese, Mn | mg | 7.6 | 1 | — | 2.28 |
| Vitamins | | | | | |
| Vitamin C, total | mg | 0.5 | 2 | — | 0.1 |
| ascorbic acid | | | | | |
| Thiamin | mg | 1.275 | 2 | — | 0.383 |
| Riboflavin | mg | 0.285 | 2 | — | 0.085 |
| Niacin | mg | 9.2 | 1 | — | 2.76 |
| Vitamin B-6 | mg | 0.6 | 2 | — | 0.18 |
| Folate, total | μg | 110 | 1 | — | 33 |
| Folic acid | μg | 0 | — | — | 0 |
| Folate, food | μg | 110 | 1 | — | 33 |
| Folate, DFE | μg | 110 | — | — | 33 |
| Vitamin A, RAE | μg | 1 | — | — | 0 |
| Carotene, beta | μg | 7 | 1 | — | 2 |
| Vitamin A, IU | IU | 11 | 1 | — | 3 |
| Vitamin E | mg | 0.8 | 1 | — | 0.24 |
| (alpha- | | | | | |
| tocopherol) | | | | | |
| Lipids | | | | | |
| Fatty acids, total | g | 4.6 | 2 | — | 1.38 |
| saturated | | | | | |
| 16:00 | g | 2.866 | 2 | — | 0.86 |
| 18:00 | g | 1.244 | 2 | — | 0.373 |
| 20:00 | g | 0.312 | 2 | — | 0.094 |
| 22:00 | g | 0.121 | 1 | — | 0.036 |
| 24:00:00 | g | 0.056 | 1 | — | 0.017 |
| Fatty acids, total | g | 5.4 | 2 | — | 1.62 |
| monounsaturated | | | | | |
| 18:1 | g | 5.276 | 2 | — | 1.583 |
| undifferentiated | | | | | |
| 18:1 c | g | 5.023 | 1 | — | 1.507 |
| 20:01 | g | 0.124 | 1 | — | 0.037 |
| Fatty acids, total | g | 38.1 | 2 | — | 11.43 |
| polyunsaturated | | | | | |
| 18:2 | g | 27.459 | — | — | 8.238 |
| undifferentiated | | | | | |
| 18:2 n-6 c,c | g | 27.358 | 2 | — | 8.207 |
| 18:2 CLAs | g | 0.202 | 1 | — | 0.061 |
| 18:3 | g | 10.024 | | | 3.007 |
| undifferentiated | | | | | |
| 18:3 n-3 c,c,c | g | 8.684 | 2 | — | 2.605 |
| (ALA) | | | | | |
| 18:3 n-6 c,c,c | g | 1.34 | 2 | — | 0.402 |
| 18:04 | g | 0.617 | 1 | — | 0.185 |
| Fatty acids, total | g | 0 | 1 | — | 0 |
| trans | | | | | |
| Cholesterol | mg | 0 | 2 | — | 0 |
| Amino Acids | | | | | |
| Tryptophan | g | 0.369 | — | — | 0.111 |
| Threonine | g | 1.269 | — | — | 0.381 |
| Isoleucine | g | 1.286 | — | — | 0.386 |
| Leucine | g | 2.163 | — | — | 0.649 |
| Lysine | g | 1.276 | — | — | 0.383 |
| Methionine | g | 0.933 | — | — | 0.28 |
| Cystine | g | 0.672 | — | — | 0.202 |
| Phenylalanine | g | 1.447 | — | — | 0.434 |
| Tyrosine | g | 1.263 | — | — | 0.379 |
| Valine | g | 1.777 | — | — | 0.533 |
| Arginine | g | 4.55 | — | — | 1.365 |
| Histidine | g | 0.969 | — | — | 0.291 |
| Alanine | g | 1.528 | — | — | 0.458 |
| Aspartic acid | g | 3.662 | — | — | 1.099 |
| Glutamic acid | g | 6.269 | — | — | 1.881 |
| Glycine | g | 1.611 | — | — | 0.483 |
| Proline | g | 1.597 | — | — | 0.479 |
| Serine | g | 1.713 | — | — | 0.514 |

In some embodiments, the amount of one or more natural components of hempseed that is modified is the amount of one or more fatty acid. The fatty acid can be saturated (16:00, 18:00, 20:00, 22:00, 24:00), monounsaturated (18.1 undifferentiated, 18:1 c), or polyunsaturated (18:2 undifferentiated, 18:2 n-6 c,c, 18:2 CLAs, 10:2 undifferentiated, 18:3 n-3 c,c,c (ALA), 18:3 n-6 c,c,c). The fatty acid can be an omega-6 or an omega-3. The percentage of the fatty acid that is removed or each of the fatty acids that are removed to the hempseed oil composition can be about 10%, 20%, 30%, 40% 50%, 60%, 70%, 80%, 90%, 95%, 99% or up to nearly 100% of the amount that is naturally occurring. The percentage of the fatty acid that is added or each of the fatty acids that are added can be about 10%, 20%, 30%, 40% 50%, 60%, 70%, 80%, 90%, 100%, 150%, 200%, 300% or more of the amount that is naturally occurring. The modification of the amount of a fatty acid or combination of fatty acids can contribute to a beneficial characteristic of the composition. For example, the removal or addition of a fatty acid or combination of fatty acids can be more effective in treating and/or preventing one or more diseases or disorders in a subject compared to an unmodified hempseed powder or oil composition or a hempseed oil composition with a different percentage of fatty acid or combination of fatty acids are that removed or added. For example, a hempseed oil composition with the removal or addition of a certain percentage of a fatty acid or combination of fatty acids can treat inflammation more effectively than an unmodified hempseed oil composition or a different hempseed oil composition. The addition of the one or more fatty acids can act synergistically with the hempseed oil.

In some embodiments, the amount of one or more natural components of hempseed that is modified is the amount of one or more omega 6 fatty acids. The percentage of omega 6 that is removed or added to the hempseed oil composition can be about 10%, 20%, 30%, 40% 50%, 60%, 70%, 80%, 90%, 95%, 99% or up to nearly 100%. The removal of omega 6 can contribute to a beneficial characteristic of the composition. For example, the removal of omega 6 can be more effective in treating and/or preventing one or more diseases or disorders in a subject compared to an unmodified hempseed oil composition or a hempseed oil composition with a different percentage of omega 6 removed. For example, a hempseed oil composition with the removal of a certain percentage of omega 6 can treat inflammation more effectively than an unmodified hempseed oil composition.

In some embodiments, the amount of one or more natural components of hempseed that is modified is the amount of one or more amino acids. The one or more amino acids can be Tryptophan, Threonine, Isoleucine, Leucine, Lysine, Methionine, Cystine, Phenylalanine, Tyrosine, Valine, Arginine, Histidine, Alanine, Aspartic acid, Glutamic acid, Glycine, Proline, and/or Serine. The percentage of the amino acid that is removed or each of the fatty acids that are removed to the hempseed oil composition can be about 10%, 20%, 30%, 40% 50%, 60%, 70%, 80%, 90%, 95%, 99% or up to nearly 100% of the amount that is naturally occurring. The percentage of the amino acid that is added or each of the amino acids that are added can be about 10%, 20%, 30%, 40% 50%, 60%, 70%, 80%, 90%, 100%, 150%, 200%, 300% or more of the amount that is naturally occurring. The modification of the amount of a amino acid or combination of amino acids can contribute to a beneficial characteristic of the composition. For example, the removal or addition of an amino acid or combination of amino acids can be more effective in treating and/or preventing one or more diseases or disorders in a subject compared to an unmodified hempseed oil composition or a hempseed oil composition with a different percentage of an amino acid or combination of amino acids are that removed or added. For example, a hempseed oil composition with the removal or addition of a certain percentage of an amino acid or combination of fatty acids can treat inflammation more effectively than an unmodified hempseed oil composition or a different hempseed oil composition. The addition of the one or more amino acid can act synergistically with the hempseed oil.

The compositions can include hempseed powder or oil with one or more amino acid removed from the composition, added to the composition, at least one amino acid removed and another amino acid added, or amino acids extracted from hempseed oil. The compositions can be used alone or in various combinations depending on intended use. The ratios of the amino acids can be adjusted based on intended use. The amino acids can be aliphatic (alanine, glycine, isoleucine, leucine, proline, valine), aromatic (phenylalanine, tryptophan, tyrosine), acidic (aspartic acid, glutamic acid), basic (arginine, histidine, lysine), hydroxylic (serine, threonine), sulphur-containing (cysteine, methionine) and/or amidic (asparagine, glutamine). The percentage of each amino acid that is removed or added to the hempseed oil composition can be about 10%, 20%, 30%, 40% 50%, 60%, 70%, 80%, 90%, 95%, 99% or up to nearly 100%. The removal or addition of one or more amino acids from the hempseed powder or oil composition can or the removal of at least one amino acid and addition of another amino acid can contribute to a beneficial characteristic of the composition.

In some embodiments, the amount of one or more natural components of hempseed that is modified is the amount of one or more cannabinoids. The percentage of the cannabinoid that is removed or each of the cannabinoids that are removed to the hempseed oil composition can be about 10%, 20%, 30%, 40% 50%, 60%, 70%, 80%, 90%, 95%, 99% or up to nearly 100% of the amount that is naturally occurring. The percentage of the cannabinoids that is added or each of the cannabinoids that are added can be about 10%, 20%, 30%, 40% 50%, 60%, 70%, 80%, 90%, 100%, 150%, 200%, 300% or more of the amount that is naturally occurring. The modification of the amount of a cannabinoid or combination of cannabinoids can contribute to a beneficial characteristic of the composition. For example, the removal or addition of a cannabinoids or combination of cannabinoids can be more effective in treating and/or preventing one or more diseases or disorders in a subject compared to an unmodified hempseed powder or oil composition or a hempseed powder or oil composition with a different percentage of cannabinoid or combination of cannabinoids are that removed or added. For example, a hempseed oil composition with the removal or addition of a certain percentage of a cannabinoid or combination of cannabinoids can treat inflammation more effectively than an unmodified hempseed oil composition or a different hempseed oil composition. The addition of the one or more cannabinoid can act synergistically with the hempseed oil.

In some embodiments, modifications to amounts are made in more that one group of lipid, protein, carbohydrate, mineral, or vitamin. For example, in one embodiment, a fatty acid can be removed from hempseed powder or oil, and an amino acid can be added. The following table illustrates potential formulas. However, in some embodiments, the amount of an addition naturally occurring component or additional naturally occurring components are modified. For example, as a non-limiting example, an embodiment of the invention could be Formula 4 plus the addition of a Cannabinoid.

TABLE 1
Formulations

| | | | Amino | Amino | | |
|---|---|---|---|---|---|---|
| | Lipid(s) | Lipid(s) | Acid(s) | Acid(s) | Cannabinoid(s) | Cannabinoid(s) |
| | Removed | Added | Removed | Added | Removed | Added |
| Lipid(s) | Formula 1 | Formula 2 | Formula 3 | Formula 4 | Formula 5 | Formula 6 |
| Removed | | | | | | |
| Lipid(s) Added | Formula 7 | Formula 8 | Formula 9 | Formula 10 | Formula 11 | Formula 12 |
| Amino Acid(s) | Formula 13 | Formula 14 | Formula 15 | Formula 16 | Formula 17 | Formula 18 |
| Removed | | | | | | |
| Amino Acid(s) | Formula 19 | Formula 20 | Formula 21 | Formula 22 | Formula 23 | Formula 24 |
| Added | | | | | | |
| Cannabinoid(s) | Formula 25 | Formula 26 | Formula 27 | Formula 28 | Formula 29 | Formula 30 |
| Removed | | | | | | |
| Cannabinoid(s) | Formula 31 | Formula 32 | Formula 33 | Formula 34 | Formula 35 | Formula 36 |
| Added | | | | | | |

Beneficial characteristics of the composition can include improved effectivity in treating and/or preventing deceases/health disorders, compared to an unmodified hempseed oil composition or a different hempseed oil composition. Beneficial characteristics of the composition can include improving or changing a flavor or fragrance or sensation of the composition, rendering the composition suitable for a specific use, function, or mode of delivery, and any combination of enhancing properties.

While hemp contains over 200 terpenes, not all are present in hempseed oil. However, the invention contemplates subtraction/removal of certain terpenes from the oil, via fractionation or otherwise, as well as addition of terpenes to the composition. Addition of one or more terpenes can be to introduce an otherwise absent terpene into the terpene profile of the oil, or to increase a given terpene's abundance, relative to other terpenes and/or other components in the oil. Likewise, removal of one or more terpenes can include either reducing a given terpene's relative abundance in the oil, or fully eliminating a given terpene from the terpene profile of the oil. Selection of which terpene(s) to add to the composition or oil, and/or which to reduce or remove from the oil, will be driven by the desired effects to be conferred or enhanced by such action.

In some embodiments, the compositions are a combination of hempseed oil, modified hempseed oil or hempseed oil extract(s) with at least one other ingredient capable of contributing a beneficial characteristic to the composition. The compositions can include a total of about 1%, 2%, 3%, 4%, 5%, 10%, 20%, 30%, 40% 50%, 60%, 70%, 80%, 90%, 95%, 99% or up to nearly 100% other ingredients.

The compositions can include one or more essential oils. The oils can act synergistically with each other, and/or with the hempseed oil, modified hempseed oil, or hempseed oil extract(s).

The compositions can include various fruit extracts and/or herbs and/or spices and/or the like. The ingredients can work synergistically with each other and/or with other ingredients, including with the hempseed oil, modified hempseed oil or hempseed oil extract(s). For example, the compositions can include one or more of Bergamot, Black Pepper, Cardamom, Cassia, Chamomile, Cilantro, Cinnamon Bark, Clary Sage, Clove, Copaiba, Coriander, Fennel, Ginger, Helichrysum, Juniper Berry, Lavender, Lemon, Lemongrass, Lime, Marjoram, Melissa, Myrrh, Oregano, Peppermint, Petitgrain, Roman, Rosemary, Spearmint, Tangerine, Thyme, Wild Orange, Ylang Ylang, and/or the like. One or more of this class of ingredients can act synergistically with other members of the class, or with other ingredients including the hempseed oil, to produce a desired effect.

For example, a composition can include one or more (or all) of Cassia, Cilantro, Cinnamon Bark, Clove, Copaiba, Lime, Marjoram, Melissa, Myrrh, Oregano, Roman Chamomile, Tangerine, and Wild Orange, in any combination, and in combination with the hempseed oil, modified hempseed oil, or hempseed oil extract(s). As another example, a composition can include one or more (or all) of Black Pepper, Cardamom, Copaiba, Fennel, Lemon, Lime, Myrrh, Oregano, Peppermint, Rosemary, and Thyme, in any combination, and in combination with the hempseed oil. As an additional example, a composition can include one or more (or all) of Black Pepper, Copaiba, Juniper Berry, Lime, Oregano, Petitgrain, Wild Orange, Ylang Ylang, in any combination, and in combination with the hempseed oil. As yet another example, a composition can include one or more (or all) of Black Pepper, Cardamom, Cassia, Cilantro, Copaiba, Coriander, Ginger, Lemon, Lemongrass, and Tangerine, in any combination, and in combination with the hempseed oil. For example, a composition can include one or more (or all) of Cardamom, Ginger, Spearmint, Myrrh. For example, a composition can include one or more (or all) of Bergamot, Cinnamon Bark, Clove, Marjoram, Thyme, Lime, Helichrysum, Black Pepper, Cinnamon Bark, Clove, Fennel, and Thyme, in any combination, and in combination with the hempseed oil. For example, a composition can include one or more (or all) of Cilantro, Helichrysum, Juniper Berry, Lemon, Thyme, wild orange, in any combination, and in combination with the hempseed oil. For example, a composition can include one or more (or all) of Bergamot, Copaiba, Helichrysum, and Marjoram. Cilantro, Clary Sage, Copaiba, Lavender, and Petitgrain, in any combination, and in combination with the hempseed oil, modified hempseed oil or hempseed oil extract(s).

The concentrations of the various other ingredients can be about 1%, 2%, 3%, 4%, 5%, 10%, 20%, 30%, 40% 50%, 60%, 70%, 80%, 90%, 95%, 99% or up to nearly 100% other ingredients. The ingredients can act synergistically with each other and/or with the hempseed oil, modified hempseed oil or hempseed oil extract(s).

The composition can be a complete vegetarian protein and lipid dietary supplement. In some embodiments, when the amino acids in the hempseed oil, modified hempseed oil or hempseed oil extract(s) are all present and in suitable ratios, the composition can provide a complete protein.

The compositions can be flavored, including with natural flavoring. The flavoring can be extracted organic flavors, or the like.

The composition can be added to food products. For example, the composition can be added to milk, juice, water, or the like. For example, the composition can be added to egg products or the like. The composition can directly supplemented to the animal to increase inflammatory agents of a final product. For example, the composition can be added to animal feed.

The composition can be in a powder formulation or an oil formulation. The composition can be ingestible. The composition can be in a topical formulation. For example, the formulation can be an external healing salve or lotion or the like.

### Methods of Making Compositions

Methods of making hempseed powder or oil compositions are provided. In some embodiments, methods can include extracting oil into different fractions for different possible intended uses. Methods can include extraction and charcoal filtration of hempseed oil into various formularies for intended use. Methods can include the breakdown of hempseed oil into a variety of basic formulas based on cell structures and intended use.

In some embodiments, methods can include reducing the hempseed to a powder by freeze-drying. In some embodiments, extractions to the powder are made using a carbon dioxide process. Relevant details can be found in U.S. patent application Ser. No. 15/451,928, the entire contents of which are hereby incorporated by reference.

In some embodiments, the final product after extractions can be a water soluble powder, oil or cream.

The resulting extracted product can be high in GLA ALA DHA and EPA.

In some methods, amino acids can be extracted from the hempseed oil. The amino acids can be removed and/or added to hempseed oil in various combinations (broken into side chains or lower level within formulas depending on intended use). The ratios of the amino acids can be adjusted based on intended use. The amino acids can include:

Aliphatic—alanine, glycine, isoleucine, leucine, proline, valine

Aromatic—phenylalanine, tryptophan, tyrosine

Acidic—aspartic acid, glutamic acid

Basic—arginine, histidine, lysine

Hydroxylic—serine, threonine

Sulphur-containing—cysteine, methionine

Amidic (containing amide group)—asparagine, glutamine

### Methods of Using Compositions for Treatment of Conditions

The compositions can be used to treat one or more conditions in a subject. The subject can be an animal subject. The subject can be a human subject. In some embodiments, one or more compositions can be used to heal and/or prevent damage to various systems of the body including but not limited to immune response, respiratory, digestive disorders, cardiovascular and heart health, nervous system disorders, cleansing formulas, and the like. For example, one or more compositions can be used for one or more of the following:

Treat and/or prevent Alzheimer's

Treat and/or prevent arthritis

Treat and/or prevent diabetes

Treat and/or prevent liver conditions,

Abdominal Fat reduction

Treat and/or prevent Muscle Damage

Improve Cognitive Abilities

Reduce Fatigue
prevents AA & Tryptophan production
causes body to store glycogen more effectively and use at a slower rate

Improve Muscle Growth-stimulates protein synthesis

Speed up Healing

Treat and/or prevent age related cognitive decline

Treat and/or prevent cardiovascular diseases

Treat and/or prevent cancer

Treat and/or prevent inflammatory diseases

Treat and/or prevent autoimmune diseases

Treat and/or prevent terminal illnesses

Treat opiate and other drug addictions

Treat ADHD

Treat insomnia

Treat MS

Reduce THC/cannabis tolerance

Treat or prevent sun burn

Treat chronic pain

Treat interstitial cystitis

Anti-aging by lengthening telomeres

Treat depression/anxiety

Treat autism spectrum disorder

Treat behavioral issues

Treat/prevent neurological problems

Balance emotional response

Increase Vision/cognitive development in infants/pregnancy

Treat macular degeneration and other vision issues

The composition can treat or prevent conditions listed in Table 2.

The hempseed product with one or more of its natural components removed, added or both can be more effective in treating and/or preventing one or more health condition compared to unmodified hempseed product or other hempseed product compositions.

For example, a method to support the immune system and its function can include administering one or more separate compositions consisting of hempseed oil, hempseed oil with one or more components extracted, hempseed oil with one or more components added, hempseed oil with one or more components extracted and one or more components added and/or varying amounts of Cassia, Cilantro, Cinnamon Bark, Clove, Copaiba, Lime, Marjoram, Melissa, Myrrh, Oregano, Roman Chamomile, Tangerine, and/or Wild Orange, in combination with hempseed oil, modified hempseed oil or hempseed oil extract(s).

For example, a method for supporting the respiratory system can include administration of seasonal and daily formulas including Black Pepper, Cardamom, Copaiba, Fennel, Lemon, Lime, Myrrh, Oregano, Peppermint, Rosemary, and Thyme, in combination with hempseed oil, modified hempseed oil or hempseed oil extract(s).

For example, a method for protecting against free radical damage can include administration of a formula including Black Pepper, Copaiba, Juniper Berry, Lime, Oregano, Petitgrain, Wild Orange, Ylang Ylang, in combination with hempseed oil, modified hempseed oil or hempseed oil extract(s).

For example, a method for adding Daily Digestion can include administering a formula including Black Pepper, Cardamom, Cassia, Cilantro, Copaiba, Coriander, Ginger, Lemon, Lemongrass, and Tangerine, in combination with hempseed oil, modified hempseed oil or hempseed oil extract(s).

For example, a method for treating stomach upset/flu/seasonal can include administering a formula including Cardamom, Ginger, Spearmint, Myrrh, in combination with hempseed oil, modified hempseed oil or hempseed oil extract(s).

For example, a method for supporting daily cardiovascular support can include Bergamot, Cinnamon Bark, Clove, Marjoram, Thyme, Lime, Helichrysum, Black Pepper, Cinnamon Bark, Clove, Fennel, and Thyme, in combination with hempseed oil, modified hempseed oil or hempseed oil extract(s).

For example, a method for a daily and quick cleanse can include administration of two formulas. One formula can be aggressive for a quick cleansing and reset and another formula can be a daily formula. The formulas can include Cilantro, Helichrysum, Juniper Berry, Lemon, Thyme, and wild orange, in combination with hempseed oil, modified hempseed oil or hempseed oil extract(s).

For example, a method for supporting the nervous system can include Bergamot, Copaiba, Helichrysum, and Marjoram. Cilantro, Clary Sage, Copaiba, Lavender, and Petitgrain, in combination with hempseed oil, modified hempseed oil or hempseed oil extract(s).

Methods using the compositions can include administering one or more compositions via specific routes or modes of administration, including but not limited to orally or topically.

Methods using the compositions can include administering one or more compositions at certain times or in certain series or other time courses. For example, the compositions can be administered regularly, or as needed, and/or seasonally.

Methods using the compositions can include administering one or more compositions in varying dosages.

The composition can include extracted organic flavors beneficial to the user for intended use. For example, strawberry leaves have been added to a composition for use in the treatment of Eczema and Rosacea.

The following is a Table that provides example formulations for treating various conditions using the hempseed oil, modified hempseed oil or hempseed oil extract(s). The Table includes optional ingredients that can be added to the hempseed oil, modified hempseed oil or hempseed oil extract(s).

TABLE 2

| | | Optional | Optional | Optional | Optional | Optional | Optional | Optional | Optional | Optional | Optional |
|---|---|---|---|---|---|---|---|---|---|---|---|
| Category | Condition | Additive 1 | Additive 2 | Additive 3 | Additive 4 | Additive 5 | Additive 6 | Additive 7 | Additive 8 | Additive 9 | Additive 10 |
| Vanity | Acne | Pot Marigold | Lavender | Hyssop | Marigold | | | | | | |
| Behavioral | ADD | Ginko | Green Oats | Ginseng | Pine Bark & | | | | | | |
| | | Biloba | | | Brahmi | | | | | | |
| Behavioral | Addiction | Radic | Peyote | | | | | | | | |
| | Alcohol | Puerariae | | | | | | | | | |
| Behavioral | Addiction | Kratom | Blue | | | | | | | | |
| | Opiate | | Trumpet | | | | | | | | |
| Auto-immune | Aids | Chinese herbs | | | | | | | | | |
| Allergy | Allergies | Marigold | Butterbar | Garlic | Stinging | Rosemary | Tumeric | | | | |
| | | | | | Nettle | | | | | | |
| Auto-immune | ALS | | | | | | | | | | |
| Neurological | Alzheimer's | Rosemary | Sage | Coconut oil | Coral | Coenzyme Q10 | Tumeric | Ginko | | | |
| | | | | | calcium | | | | | | |
| Pain | Analegic | Peppermint | | | | | | | | | |
| Anti-Aging | Anti Aging | French | Ginseng | Cordyceps | Rhodiola | Aswaganda | Holy Basil | tumeric | | | |
| | | Lilac | | | | | | | | | |
| AntiBacterial | Anti-biotic | Honey | Cardamom | Sweet | Garlic | Sweet | | | | | |
| | | | | Marjoram | | Plaintain | | | | | |
| AntiBacterial | Antibiotic | Ginger | Clover | Echinacea | Goldenseal | Clove | | | | | |
| Internal | Anti-Inflammatory | Capsaicin | Cardamom | Frankincense | Tumeric | Royal Jelly | GreenTea | Black Pepper | Resveratrol | White | Maritime |
| | | | | | | | | | | Willow | Pine Bark |
| | | | | | | | | | | Bark | |
| Skin | Anti-Inflammatory | Lovage | Licorice Rot | Bromelain | Willow Bark | Witch Hazel | Chamomile | Yarrow | Oak bark | | |
| AntiBacterial | AntiSeptic | Thyme | Lavender | Yarrow | Oregano | Hussop | | | | | |
| Behavioral | Anxiety | Aniseed | Sweet Cicely | Feverfew | Kava | | | | | | |
| Arthritis | Arthritis | FeverFew | | | | | | | | | |
| Auto-immune | Asthma | Thyme | Sage | Peppermint | Lavender | Clove | Eucalptus | | | | |
| Skin | Astringent | Broadleaf | Yarrow | Witch | | | | | | | |
| | | Plantan | | Hazel | | | | | | | |
| Heart | Atherosclerosis | Garlic + Q10 | | | | | | | | | |
| Health | | | | | | | | | | | |
| Anti-Fungal | Athletes | Great | Tea Tree | Garlic | Baking soda | Iodine | | | | | |
| | Foot | Mullen | | | | (walnt) | | | | | |
| Pain | Atrhritic/Joint | GLA | Capsaicin | Devils Claw | Marigold | | | | | | |
| | Pain | | | | | | | | | | |
| Behavorial | Autism | Probiotics | Greens | | | | | | | | |
| General | Autoimmunity | Echinacea | Royal Jelly | | | | | | | | |
| Health | | | | | | | | | | | |
| Pain | Back/neck | Devils Claw | | | | | | | | | |
| | pain | | | | | | | | | | |
| Skin | Bloating | Capsaicin | | | | | | | | | |
| Detox | Blood | Burdock | | | | | | | | | |
| | Cleaser | Root | | | | | | | | | |
| Bone | Bone | Comfrey | | | | | | | | | |
| | Health | | | | | | | | | | |
| Neurological | Brain Aging | Rosemary | | | | | | | | | |
| Cancer | Breast | Garlic | Echinacea | Tumeric | Burdock | Carotenoids | Green Tea | | | | |
| | Cancer | | | | | | | | | | |
| hormone | Breast pain | | | | | | | | | | |
| Skin | Breath | Parsley | | | | | | | | | |
| | Freshner | | | | | | | | | | |
| general | Bronchitis/cough | Agrimony | Aniseed | Red Clover | Yarrow | Thyme | Great | | | | |
| Health | | | | | | | Mullen | | | | |
| Skin | Burns | Aloe | Echinacea | Stevia | | | | | | | |
| Cancer | Cancer | Cardamom | Tumeric | Green tea | black pepper | oregano | Strawberry | | | | |
| Regeneration/ | Cell Growth | Broadleaf | | | | | | | | | |
| Growth | | Plantain | | | | | | | | | |
| Pain | Chronic | Tumeric | Resveratrol | THC | | | | | | | |
| | Pain | | | | | | | | | | |
| Circulation | Circulation | Cardamom | Marigold | Elderberry | | | | | | | |
| | support | | | | | | | | | | |
| Anti-Viral | Colds | Echinacea | Catmint | Red Clover | Sweet Violet | Yatrrow | Ginger | Olive Leaf | | | |
| Behavorial | Colic | Chamomile | Stevia | | | | | | | | |
| GI | Constipatatoin | Aloe Vera | Milk Thistle | | | | | | | | |
| Hair | Cover Grey | Marigold | | | | | | | | | |
| | Hair | | | | | | | | | | |
| Neurological | CTE | Parsley | Melatonin | | | | | | | | |
| Auto-immune | Cystic | | | | | | | | | | |
| | Fibrosis | | | | | | | | | | |
| Hormone | Cysts | | | | | | | | | | |
| Anti-Viral | Decongstant | Thyme | Great Mullen | Aniseed | Honey | Eucalyptus | Peppermint | Oregano | | | |
| Anti-Viral | Defend | Astragalus | Cats Claw | Lemon | | | | | | | |
| | against | | | Balm | | | | | | | |
| | Viruses | | | | | | | | | | |
| Neurological | Dementia | Sage | | | | | | | | | |
| Behavorial | Depression | Sage | Ashwagandha | | | | | | | | |
| Skin | Dermatitis | Cardamom | Hydrocortisone | | | | | | | | |
| Detox | Detox | Red Clover | Parsley | Sweet Violet | | | | | | | |
| Animal | Deworming | Tansy | Castor oil | | | | | | | | |
| HJormone | Diabetes | French | | | | | | | | | |
| | | Lilac | | | | | | | | | |
| GI | Diarrhea | Broadleaf | Winter | | | | | | | | |
| | | Plantan | Savory | | | | | | | | |
| GI | Digestive | Sweet | Cinnamon | | | | | | | | |
| | Aid | Marjoram | | | | | | | | | |
| GI | Digestive | Pot | Aniseed | | | | | | | | |
| | Upset | Marigold | | | | | | | | | |
| GI | Diruetic | GreenThread | | | | | | | | | |
| Skin | Dry Scalp | Coconut oil | Tea Tree | Aloe Vera | Apple Cider | Witch | Baking | Bananas | | | |
| | | | | | Vinegar | Hazel | Soda + | | | | |
| | | | | | | | Olive Oil | | | | |
| Skin | Dry Skin | Aloe Vera | Chamomile | Calendula | Horsetail | Peppermint | | | | | |
| Hormone | Dysmenorrhea | Sage | | | | | | | | | |
| Anti-Viral | Ear | Mullein + | Olive oil | Tea tree oil | | | | | | | |
| | Infections | Garlic | | | | | | | | | |
| Skin | Eczema | Marigold | Coconut oil | Yarrow | | | | | | | |
| Swelling | Edema | Catmint | FeverFew | | | | | | | | |
| hormone | Endometrosis | | | | | | | | | | |
| Daily | Energy | Ginseng | Cordyceps | | | | | | | | |
| General | Fatigue | Fo-ti | | | | | | | | | |
| Health | | | | | | | | | | | |
| Viral | Fever | Catmint | Marigold | | | | | | | | |
| Auto-immune | Fibromyalgia | Lavender | Peppermint | 5-http | Sage | Magnesium | St Johns | Melatonin | | | |
| | | | | | | | Wort | | | | |
| General | Flatulance | Aniseed | Catmint | Lovage | Winter | | | | | | |
| Health | | | | | Savory | | | | | | |
| Anti-Viral | Flu | Echinacea | Hyssop | Sweet violet | Olive Leaf | | | | | | |
| General | Food borne | Coriander | | | | | | | | | |
| Health | pathogens | | | | | | | | | | |
| Infections | Fungal | Hyssop | Garlic | | | | | | | | |
| | infections | | | | | | | | | | |
| Anti-Viral | Gastric | Licorice | Honey | Garlic | Tumeric | Aloe vera | | | | | |
| | Ulcers | Root | | | | | | | | | |
| GI | Gastrointestinal | Comfrey | Cardamom | Catmint | GreenThread | Lovage | | | | | |
| Pain | General | White | Peppermint | | | | | | | | |
| | | willow | | | | | | | | | |
| Dental | Gingivitis | Peppermint | Comfrey | | | | | | | | |
| Arthritis | Gout | Broadleaf | Cherry | Magnesuim | Ginger | Celery | Nettle | Dandelion | Milk Thistle | | |
| | | Plantain | Juice | | | | | | | | |
| Pain | Headache | Catmint | FeverFew | Marigold | Sweet | | | | | | |
| | | | | | Violet | | | | | | |
| General | Heal | Echinacea | Echinacea + | Alfalfa | | | | | | | |
| Health | Infections | | Oregon | | | | | | | | |
| | | | Grape Root | | | | | | | | |
| Cardiovascular | Heart Disease | Hawthorn | Garlic | Ginko Biloba | | | | | | | |
| Heart | High | | | | | | | | | | |
| Health | | | | | | | | | | | |
| Hormone | Heavy | Comfrey | | | | | | | | | |
| | menstrual | | | | | | | | | | |
| | flow | | | | | | | | | | |
| General | Hemorrhiods | Benzocaine | Witch Hazel | Apple Cider | Psyllium | Aloe Vera | Tea Tree | Epson Salt | | | |
| Health | | | | Vinegar | Husk | | Oil | | | | |
| Anti-Viral | Herpes | Olive Leaf | Tea Tree | Peppermint | Anise | Lemon | Oregano | Thyme | Ginger | | |
| | | | | | | Balm | | | | | |
| Blood | High | Sage | Sweet | Stevia | | | | | | | |
| Pressure | BloodPressure/ | | Cicely | | | | | | | | |
| | hypertension | | | | | | | | | | |
| Blood | High | Coriander | | | | | | | | | |
| Pressure | Triglycerides | | | | | | | | | | |
| Pain | HIV | Capsaicin | | | | | | | | | |
| Hormone | Hormone | | | | | | | | | | |
| | Regulation | | | | | | | | | | |
| Behavorial | Hyperactivity | Ginko Biloba | Brahmi | Gotu Kola | | | | | | | |
| Skin | HyperHydrosis | Sage | | | | | | | | | |
| Blood | Hypotension/ | Hawthorn | Celery seed | | | | | | | | |
| Pressure | low blood | | | | | | | | | | |
| | Pressure | | | | | | | | | | |
| Auto-immune | Immune | Gingseng | Astragalus | | | | | | | | |
| | deficiencies | | Root | | | | | | | | |
| Sexual | Impotence | Cardamom | | | | | | | | | |
| Pregnancy | Increases | Aniseed | | | | | | | | | |
| | milk supply | | | | | | | | | | |
| GI | Indegestion | Stevia | | | | | | | | | |
| Infections | Infection | Great | Garlic | | | | | | | | |
| | | Mullen | | | | | | | | | |
| Hormone | Infertility | Aloe Vera | | | | | | | | | |
| Skin | Insect Bites | Echinacea | Broadleaf | | | | | | | | |
| | | | Plantain | | | | | | | | |
| Diabetic | Insulin | Coriander | | | | | | | | | |
| | Production | | | | | | | | | | |
| Bladder | Interstistal | Marshmallow | Aloe Vera | | | | | | | | |
| | Cystsis | Root | | | | | | | | | |
| GI | Intestinal | Cinnamon | Garlic | Clove | | | | | | | |
| | Bacteria | | | | | | | | | | |
| GI | Intestinal | Aloe Vera | | | | | | | | | |
| | Disorders | | | | | | | | | | |
| GI | Irritable | Artichoke | | | | | | | | | |
| | Bowel | leaf | | | | | | | | | |
| | Disease | | | | | | | | | | |
| Skin | Itching | Yarrow | | | | | | | | | |
| Organ | Kidney | Broadleaf | Dandelion | | | | | | | | |
| | Disease | Plantain | | | | | | | | | |
| Behavioral | Learnig | California | catnip | Ginko | | | | | | | |
| | Disorders | Poppy | | Biloba | | | | | | | |
| Cancer | Leukemia | | | | | | | | | | |
| Muscle | Ligament | Comfrey | | | | | | | | | |
| | Injury | | | | | | | | | | |
| Organ | Liver | Alfalfa | Blue | Dandelion | Milk Thistle | Tumeric | | | | | |
| | support | | Trumpet | | | | | | | | |
| Animal | Livestock | Alfalfa | Red Clover | | | | | | | | |
| Weight | Low | | | | | | | | | | |
| | Metabolism | | | | | | | | | | |
| Stress | Lower | Holy Basil | | | | | | | | | |
| | cortisol | | | | | | | | | | |
| Diabetic | Lowers | Stevia | | | | | | | | | |
| | blood sugar | | | | | | | | | | |
| Heart | Lowers | Alfalfa | Policosanol- | ALA | Artichoke | Hawthorne | garlic | | | | |
| Health | cholesterol | | sweetener | | Extract | | | | | | |
| Auto-immune | Lupus | Tumeric | Ginger | Coconut oil | Holy Basil | GreenTea | | | | | |
| Dietary | Malnutrition | Rosemary | | | | | | | | | |
| Behavioral | Memory | Ginko Biloba | Ashwagandha | Periwinkle | Peppermint | Blueberry | Rosemary | Ginseng | | | |
| | Enhancer | | | | | | | | | | |
| Hormone | Menopause | Sage | | | | | | | | | |
| Hormone | Mestasis | Aloe Vera | | | | | | | | | |
| Injury | Minor Cuts | Thyme | Chamomile | | | | | | | | |
| hormone | Morning | Alfalfa | | | | | | | | | |
| | Sickness | | | | | | | | | | |
| Anti-viral | Mouth | Aloe | Chamomile | Echinacea | Myrrh | | | | | | |
| | Sores | | | | | | | | | | |
| Neurological | Multiple | Peppermint | | | | | | | | | |
| | Sclerosis | | | | | | | | | | |
| Muscle | Muscle | | | | | | | | | | |
| | Cramps | | | | | | | | | | |
| GI | Nausea | Aniseed | Capsaicin | | | | | | | | |
| Pain | Nerve Pain | Capsaicin | | | | | | | | | |
| Pain | Pain Relief | FeverFew | THC | | | | | | | | |
| Pain | Pain Relief | Broadleaf | THC | Lovage | | | | | | | |
| | | Plantan | | | | | | | | | |
| Dental | Periodontitis | Evening | Cardamom | | | | | | | | |
| | | Primrose | | | | | | | | | |
| AntiBacterial | Pink Eye | Calendula | Tea Tree | Myyrh | Eucalptus | Clove | Lavender | Roman | | | |
| | | | | | oil | | | Chamomile | | | |
| Pain | Post Cancer | Capsaicin | | | | | | | | | |
| | Surgery | | | | | | | | | | |
| AntiBacterial | Prevent | Aloe | Hyssop | Calendula | Garlic | | | | | | |
| | infections | pulcherrima | | | | | | | | | |
| | | roots | | | | | | | | | |
| General | Prevent bug | Tansy | | | | | | | | | |
| Health | bites | | | | | | | | | | |
| General | Protein | Alfalfa | BCAA | | | | | | | | |
| Health | Supplement | | | | | | | | | | |
| Skin | Psoriasis | Capsaicin | Dead Sea | Tumeric | Tea Tree | | | | | | |
| | | | Salt | | Oil | | | | | | |
| general | Relaxant | Sweet | Chamomile | Lavender | | | | | | | |
| Health | | Violet | | | | | | | | | |
| Neurological | Schizophrenia | Salycilic | | | | | | | | | |
| | | Acid | | | | | | | | | |
| Allergy | skin rash | Marigold | Olive oil | Baking soda | | | | | | | |
| Skin | Skin Sores | Broadleaf | Olive oil | Baking soda | Aloe vera | Chamomile | Neem | | | | |
| | | Plantain | | | | | | | | | |
| Skin | Skin | Ginger | Peppermint | Chamomile | | | | | | | |
| | tightening | | | | | | | | | | |
| Sleep | Sleep Assist | Lavender | Agrimony | Sweet violet | | | | | | | |
| Anti-viral | Sore Throat | Comfrey | Aniseed | Winter | Benzocaine | Apple cider | Lemon | Ginger | | | |
| | | | | Savory | | | | | | | |
| Organ | Spleen | Dandelion | | | | | | | | | |
| | Health | | | | | | | | | | |
| Infections | Staph | Usnea | | | | | | | | | |
| GI | Stomach | Catmint | Winter | Yarrow | | | | | | | |
| | Ache | | Savory | | | | | | | | |
| GI | Stomach | Comfrey | Lovage | | | | | | | | |
| | Ulcers | | | | | | | | | | |
| Anti-Viral | Strep Throat | Usnea | Peppermint | Lavender | Echinacea | Eucalyptus | Ginger | Lemon | Thyme | Bitter | |
| Stress | Stress | Rhodiola | Ashwagandha | | | | | | | Orange | |
| | Reduction | | | | | | | | | | |
| Heart | Stroke | Pot | | | | | | | | | |
| Health | | Marigold | | | | | | | | | |
| Skin | Sun Burn | Benzocaine | Aloe Vera | | | | | | | | |
| Hormone | Thyroid | Pot | | | | | | | | | |
| | Function | Marigold | | | | | | | | | |
| General | Tissue | Comfrey | | | | | | | | | |
| Health | Repair | | | | | | | | | | |
| Dental | Toothache | Clove | | | | | | | | | |
| Pain | Toothnumbing | Benzocaine | | | | | | | | | |
| Pain | Topical | Clove | | | | | | | | | |
| | Pain | | | | | | | | | | |
| Detox | Toxin flushing | Alfalfa | | | | | | | | | |
| Outdoor/skin | Treat bug bites | Winter | | | | | | | | | |
| | | Savory | | | | | | | | | |
| Cancer | Tumor | Marigold | | | | | | | | | |
| | Development | | | | | | | | | | |
| GI | Ulceratative | Aloe | Butyrate | Tormentil | Wheatgrass | Tumeric | | | | | |
| | Colitis | | | Extract | | | | | | | |
| Anti-Viral | Urinary Tract | Alfalfa | Sweet | GreenThread | Lovage | Marshmellow | Uva Ursi | Cranberry | | | |
| | Infections | | Cicely | | | Root | | | | | |
| Circulation | Varicose Veins | | | | | | | | | | |
| Viral | Viruses | Echinacea | | | | | | | | | |
| Eye care | Vision Disorders | | | | | | | | | | |
| GI | Vomiting/Nausea | Alfalfa | | | | | | | | | |
| Skin | Warts | Great | | | | | | | | | |
| | | Mullen | | | | | | | | | |
| Skin | Wrinkles | | | | | | | | | | |
| Anti-Viral | Yeast Infections | Usnea | Greek Yogurt | Boric Acid | Oregano | Coconut oil | Tea Tree | Garlic | Apple Cider | | |
| | | | | | | | Oil | | Vinegar | | |

### Examples

### Effectivity of Modified Hempseed Oil Compositions

The effectiveness of the modified hempseed oil compositions to treat inflammation is tested. The effectiveness of a modified hempseed oil composition as described herein is compared to unmodified hempseed oil composition. Efficacy is tested by evaluating subjects treated with modified hempseed oil compositions. For example, subjects are evaluated using DNA markers for inflammation. For example, subjects are evaluated using standard blood serum tests for inflammatory response and omega 3:6 ratios.

Tests are performed depending on the treatment. For example, for subjects treated for dermatological conditions, skin tones, itchiness from rash/diseases, healing rate & free radical protection can be tested.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the at least two active ingredients selected for the pest control composition, the target pest, and the amounts of the various ingredients present in the pest control composition. Various embodiments of the invention can specifically include or exclude any of these variations or elements.

In some embodiments, the numbers expressing quantities of ingredients, properties such as molecular weight, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Variations on embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, to the extent that any references have been made to patent documents and other printed publications throughout this specification, each of the cited patent documents and other printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

What is claimed is:

1. A composition comprising modified hempseed oil, wherein a modification is made to hempseed oil, wherein the modification comprises removal of one or more naturally occurring components of hempseed oil, addition of one or more naturally occurring hempseed oil, or removal of at least one component and addition of another component, wherein the modification is capable of contributing to a beneficial characteristic of the composition.

2. The composition of claim 1 wherein the one or more naturally occurring components of hempseed oil is selected from a lipid, an amino acid or a cannabinoid.

3. The composition of claim 2, wherein the lipid is Omega 6 fatty acid, wherein Omega 6 is removed from hempseed oil.

4. The composition of claim 1 wherein the beneficial characteristic of the composition is effectivity in the treatment of a health condition.

5. The composition of claim 4 wherein the health condition is an inflammatory disease.

6. (canceled)

7. (canceled)

8. The composition of claim 4, wherein the enhancing ingredients are one or more of the group of all ingredients cited in the text of this application, and/or any similar or related ingredients.

9. A method of ameliorating a condition by administering the composition of claim 4.

10. A method of making the composition of claim 1, comprising selecting a hempseed oil or an extract or modification thereof;
selecting at least one enhancing ingredient based upon a desirable property of the final composition;
combining the enhancing ingredient(s) with the hempseed oil or extract or modification thereof;
optionally adding a non-enhancing (inactive) ingredient; and
obtaining the composition of claim 1.
